(19) United States
(12) Patent Application Publication
(10) Pub. No.: US 2018/0154184 A1
(43) Pub. Date: Jun. 7, 2018

(54) APPLICATION OF HIGH-INTENSITY FOCUSED ULTRASOUND SYSTEM TO TREATMENT OF ESSENTIAL HYPERTENSION

(71) Applicant: NANJING KHONS MEDTECH CO., LTD., Nanjing, Jiangsu (CN)

(72) Inventors: Xiangqing KONG, Nanjing (JP); Younian KONG, Nanjing (CN)

(73) Assignee: NANJING KHONS MEDTECH CO., LTD., Nanjing, Jiangsu (CN)

(21) Appl. No.: 15/574,555

(22) PCT Filed: Jun. 24, 2016
(86) PCT No.: PCT/CN2016/087083

§ 371 Date: Nov. 16, 2017

(30) Foreign Application Priority Data
Dec. 17, 2015 (CN) 201510956161.2

(51) Int. Cl. A61N 7/02

(52) U.S. Cl. CPC A61N 7/02; A61N 2007/0008

(57) ABSTRACT

An application of a high-intensity focused ultrasound system to treatment of essential hypertension. A treatment process includes: conducting ultrasonic positioning measurement on both sides of perirenal fat tissue of a patient, setting a therapeutic window and parameters of the system, and setting corresponding power parameter of the system, so as to make local temperature of the tissue during treatment reach 40-70° C., wherein treatment portions are both sides, and treatment scope is one third to all of the whole tissue; starting the system to treat one side of the tissue according to the set parameters and then the other. By treating a secondary center for regulating the activity of a whole-body sympathetic system, the activity of the whole-body sympathetic system can be reduced, so that the blood pressure level of the patient can be reduced, and fewer kinds and smaller dosage of antihypertensive drugs can be taken or ceased.

### TECHNICAL FIELD

The present invention belongs to the field of hypertension treatment, and particularly relates to application of a high-intensity focused ultrasound system to treatment of essential hypertension.

### RELATED ART

A high-intensity focused ultrasound (HIFU) treatment system (HIFU treatment system for short hereinafter) is mainly composed of a power source, a treatment control system, a positioning and real-time evaluating system, a motion control system and the like. The treatment principle of the HIFU treatment system is that due to the heat effect generated by high-intensity ultrasound focused in biological tissue, coagulative necrosis of tissue located in a focal region is realized instantaneously, no obvious damage is caused to tissue beyond the focal region, and the coagulative necrosis tissue can be gradually absorbed or scarred. The new technique of local treatment is currently limited to treatment of malignant and benign solid tumors of tissue and organs, and multiple clinical tests prove that the treatment system can effectively cause coagulative necrosis of tumor tissue, reduce tumor volume, increase the success rate of surgical removal of tumors, and achieve palliative treatment of tumors.

The HIFU treatment system not only has a tumor tissue removing function, but has the same effect on non-tumor in vivo biological tissue, however, difference exists in adopted power and local temperature control of target tissue. Application in this aspect is still blank currently.

Essential hypertension is one of major chronic diseases endangering human health and life currently, and the incidence rate of the essential hypertension becomes higher and higher as economy develops and the average life span becomes longer. According to the cardiovascular disease report of China in 2006, the number of hypertension patients in China has reached 200 million. It is estimated that by 2025, the hypertension patients across the world will exceed 1.5 billion, including 300 million of Chinese patients.

The essential hypertension can develop from no symptom to organ damage step by step. The awareness rate of patients about the disease is usually low in the stage when no symptom appears. The blood pressure control up-to-standard condition of patients after confirmed diagnosis is still not optimistic. Though both China and America put forward in their own hypertension guides that reducing blood pressure to a normal level as early as possible is the basic requirement of optimizing blood pressure management, it is estimated that the blood pressure control up-to-standard rate of hypertension patients in America is only about 35%, and 8% in China. Therefore, the number of hypertensive complication patients also increases day by day due to the high incidence rate, the low awareness rate and the low control rate of hypertension, and hypertensive complication patients mainly include apoplexy (vascular thrombosis or in situ thrombosis), heart failure (contraction type and relaxation type) and progressive renal failure patients, resulting in great social and economic burden.

Currently, the essential hypertension is mainly treated through long-term oral taking of drugs. There are various kinds of hypertension drugs for treating the hypertension, however, the blood pressure level of a large number of patients is still not up to standard. Besides, drug interaction and side effects caused by massive drug taking can affect the decision of a physician on whether treatment continues, or affect the compliance of patients to a treatment scheme. Past studies show that the blood pressure up-to-standard rates of hypertension patients with high, medium and low compliance are 43%, 34% and 33% respectively. The proportion of patients in foreign countries with better compliance to orally taken antihypertensive drugs reaches 50-84%, while only 30% or so in China.

Therefore, it will benefit patients, physicians and a medical system to provide a non-medicine treatment method as replacement therapy. At present, there are mainly two kinds of non-medicine methods for treating hypertension in the world, both of which are invasive. One is transcatheter renal artery sympathetic denervation (RSD), and the other is arteriovenous anastomosis using an ROX device. The principle of RSD is that radiofrequency catheter ablation of renal artery sympathetic nerves is conducted in the hope of breaking off the vicious circle between hypertension and renal damage, so as to reduce blood pressure. A recently released SYMPLICITYIII test result shows that compared with a sham-operated group, the high blood pressure control proportion and the blood pressure reduction degree of patients in a transcatheter renal artery sympathetic denervation surgical group do not show obvious statistic difference, indicating that the treatment method is ineffective. The principle of ROX device surgery is that a stable fistula is formed between arteries and veins by means of an interventional method to form left-right diversion at the position of a peripheral blood vessel, so that the capacity of an arterial system is reduced and arterial compliance is improved. The preliminary clinical trial of the device is proved to be remarkably effective, however, no clinical controlled trial of a sham-operated group is conducted yet, and more severe clinical complications, like hematomas, dissection, venous thrombosis, device falling-off and right ventricular volume overload, occur to postoperative patients.

### SUMMARY

The present invention aims to provide application of a high-intensity focused ultrasound system to treatment of essential hypertension based on the prior art.

The present invention also aims to provide a method for treating the essential hypertension by means of the high-intensity focused ultrasound system.

The objective of the present invention can be realized through following measures:

according to the application of the high-intensity focused ultrasound system to the treatment of the essential hypertension,

a secondary center (both sides of perirenal fat tissue), discovered by the inventor, for regulating the activity of a whole-body sympathetic system is treated by means of the high-intensity focused ultrasound system, so as to reduce the activity of the whole-body sympathetic system, in this way, the blood pressure level of an essential hypertension patient can be reduced, side effects caused by oral taking of antihypertensive drugs are avoided, and the risk of cardiovascular complications is reduced.

A process for realizing the application provided by the present invention, namely the method for treating the essential hypertension by means of the high-intensity focused ultrasound system comprises the following steps of:

(1) conducting ultrasonic positioning measurement on a treatment target, namely both sides of perirenal fat tissue of an essential hypertension patient, and measuring the sagittal length, the sagittal width, the horizontal width and the height of the perirenal fat tissue;

(2) setting a therapeutic window and corresponding parameters of the high-intensity focused ultrasound system according to weight, height and subcutaneous fat thickness parameters of the patient, so as to make local temperature of the perirenal fat tissue during treatment reach 40-70° C., treatment portions are both sides of the perirenal fat tissue, and a treatment scope is one third to all of the whole tissue;

(3) after setting the therapeutic window and the corresponding parameters, starting the high-intensity focused ultrasound system to treat one side of the perirenal fat tissue according to the set parameters; and

(4) after treating one side of the perirenal fat tissue, treating the other side of the perirenal fat tissue according to the same method and parameters.

In the treatment process and method, the treatment target is both sides of the perirenal fat tissue; preferably, the treatment target is both sides of lower perirenal fat tissue, and the preferred target can reduce damage to adjacent tissue (kidney, blood vessels, ureters and other structures) during treatment as much as possible while the treatment purpose is realized.

Lower perirenal fat in the present invention refers to the fat tissue, located below the lower portion of kidney, in the perirenal fat tissue.

In one preferred scheme, time for treating one side of the treatment target is 9-70 min, preferably 10-60 min, and most preferably 20-40 min. Treatment time in the present invention means the total ultrasonic emission time of the high-intensity focused ultrasound system.

In therapeutic processes of step 3 and step 4, local temperature of the treatment target perirenal fat tissue is made to reach 40-70° C., preferably 45-60° C. by means of the high-intensity focused ultrasound system. In the process, the therapeutic window and the corresponding parameters which are set before can also be adjusted properly according to a specific treatment process or a measurement result on the temperature of the target obtained by means of various devices, so as to make local temperature of the treatment target meet a predetermined requirement, or meet the requirement faster or better.

During treatment, local temperature of the treatment target perirenal fat tissue is made to reach 40-70° C., preferably 45-60° C. by means of the high-intensity focused ultrasound system.

In one preferred scheme, the treatment scope of the treatment target is one third to two thirds of both sides of the perirenal fat tissue.

The therapeutic window of the high-intensity focused ultrasound system comprises treatment spots defining the treatment scope of the high-intensity focused ultrasound system, the size of each treatment spot, and the relative positions of the treatment spots. The corresponding parameters of the high-intensity focused ultrasound system include but not limited to the power, the emission time, the emission time interval and the number of emission times of the high-intensity focused ultrasound system.

The essential hypertension patient does not eat or drink anything 3 h before treatment, wears loose patient dress, adopts a proper posture before treatment, and keeps limbs fixed during treatment.

The application and the method have the advantages that

(1) by treating the secondary center, discovered by the inventor, for regulating the activity of the whole-body sympathetic system by means of the high-intensity focused ultrasound system, the activity of the whole-body sympathetic system can be reduced, so that the blood pressure level of the essential hypertension patient can be reduced, and the patient can take fewer kinds and a smaller dosage of antihypertensive drugs or even stop taking the drugs;

(2) by controlling the level of blood pressure and/or blood glucose of the patient, the risk of cardiovascular complications is reduced; and

(3) by treating the secondary center, the activity of the whole-body sympathetic system is reduced, insulin resistance is reduced, whole-body fibrosis is reduced, and other diseases, such as sudden cardiac death, ventricular arrhythmias of various types and heart failure, characterized in activity abnormity of the sympathetic system might be treated too.

### BRIEF DESCRIPTION OF THE DRAWINGS

FIG. 1 is a diagram showing how to treat essential hypertension by means of a high-intensity focused ultrasound system according to the present invention.

FIG. 2 is a diagram of both sides of perirenal fat tissue and lower perirenal fat.

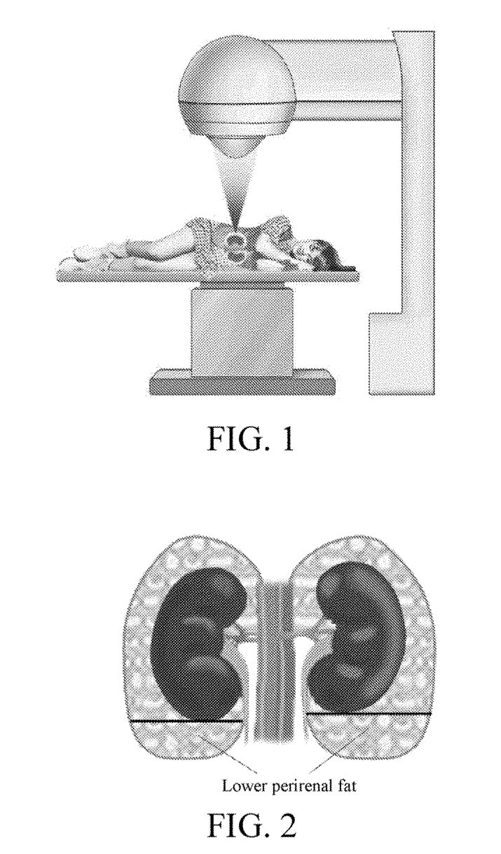

### DETAILED DESCRIPTION

Further explanation is made with reference to the drawings and embodiments, however, the protection scope of the prevent invention is not limited to the embodiments.

### Embodiment 1: Treatment of Mild to Moderate Essential Hypertension

Female patient, 64 years old, being 170 cm in height and 80 kg in weight, taking irbesartan 75 mg/d before operation, clinical blood pressure being 140/80 mmHg, average blood pressure within 24 h being 130/70 mmHg.

Before surgery, ultrasonic positioning measurement was conducted on the right side of perirenal fat tissue of the patient, and the sagittal length, the sagittal width, the horizontal width and the height of lower perirenal fat were measured to be 3.6 cm, 4.1 cm, 2.5 cm and 3.9 cm respectively. A therapeutic window scope of a high-intensity focused ultrasound system was set to have 78 spots, the size of each spot was 25.7×63.5×13.5 mm³, and the distance between every two adjacent spots was 3.5 mm in line spacing, 3.5 mm in step pitch and 6.0 mm in layer spacing; the power parameter of the high-intensity focused ultrasound system was set to be 140 W, emission time was 699 ms, the interval was 10 ms, the number of emission times was 40, local temperature of the perirenal fat tissue was made to reach 48° C. during treatment, the treatment scope was one third of the whole tissue, and the total emission time was 36 min 20 s; after treatment of one side is ended, the other side was treated according to the same method and parameters.

The patient stopped taking antihypertensive drugs after treatment. In one-month follow-up, the clinical blood pressure being 126/74 mmHg, the average blood pressure within 24 h being 128/73 mmHg; in six-month follow-up, the clinical blood pressure being 124/70 mmHg, and the average blood pressure within 24 h being 125/67 mmHg.

### Embodiment 2: Treatment of Severe Hypertension

Male patient, 42 years old, being 174 cm in height and 88 kg in weight, taking nifedipine controlled release tablets 60 mg/d, irbesartan 150 mg/d and hydrochlorothiazide 12.5 mg/d before operation, clinical blood pressure being 180/110 mmHg, average blood pressure within 24 h being 158/104 mmHg.

Before surgery, ultrasonic positioning measurement was conducted on the right side of perirenal fat tissue of the patient, and the sagittal length, the sagittal width, the horizontal width and the height of lower perirenal fat were measured to be 7.7 cm, 5.2 cm, 4.5 cm and 4.8 cm respectively. The therapeutic window scope of the high-intensity focused ultrasound system was set to have 74 spots, the size of each spot was 21.6×30.4×17.0 mm³, and the distance between every two adjacent spots was 3.0 mm in line spacing, 3.5 mm in step pitch and 6.0 mm in layer spacing; the power parameter of the high-intensity focused ultrasound system was set to be 150 W, emission time was 699 ms, the interval was 10 ms, the number of emission times was 40, local temperature of the perirenal fat tissue was made to reach 55° C. during treatment, the treatment scope was two thirds of the whole tissue, and the total emission time was 34 min 29 s; after treatment of one side is ended, the other side was treated according to the same method and parameters.

The patient stopped taking hydrochlorothiazide after treatment. In one-month follow-up, the clinical blood pressure being 146/94 mmHg, the average blood pressure within 24 h being 141/98 mmHg; in six-month follow-up, the clinical blood pressure being 138/90 mmHg, and the average blood pressure within 24 h being 135/88 mmHg.

### Embodiment 3: Human Trials

We have conducted 30 human trials. The corresponding therapeutic window and parameters of each object were adjusted according to the method of embodiment 1 and embodiment 2 for treatment.

The 30 preliminary human trials included refractory hypertension and hypertension patients accompanied by cardio-cerebrovascular complications, the blood pressure of all the patients dropped steadily without exception after surgery, the patients took fewer kinds and a smaller dosage of antihypertensive drugs, and ten patients can even stop taking the drugs completely. Now we have conducted follow-up for over 6 months and found that the blood pressure of the patients is steady, and no rebound situation occurred.

1. Application of a high-intensity focused ultrasound system to treatment of essential hypertension.

2. The application according to claim 1, wherein a treatment process comprises the following steps of:
(1) conducting ultrasonic positioning measurement on a treatment target, namely both sides of perirenal fat tissue of an essential hypertension patient, and measuring the sagittal length, the sagittal width, the horizontal width and the height of the perirenal fat tissue;
(2) setting a therapeutic window and corresponding parameters of the high-intensity focused ultrasound system according to weight, height and subcutaneous fat thickness parameters of the patient, so as to make local temperature of the perirenal fat tissue during treatment reach 40-70° C., treatment portions are both sides of the perirenal fat tissue, and a treatment scope is one third to all of the whole tissue;
(3) after setting the therapeutic window and the corresponding parameters, starting the high-intensity focused ultrasound system to treat one side of the perirenal fat tissue according to the set parameters; and
(4) after treating one side of the perirenal fat tissue, treating the other side of the perirenal fat tissue according to the same method and parameters.

3. The application according to claim 2, wherein the treatment target is both sides of lower perirenal fat tissue.

4. The application according to claim 2, wherein time for treating one side of the treatment target is 9-70 min.

5. The application according to claim 2, wherein local temperature of the treatment target is made to reach 45-60° C. during treatment.

6. The application according to claim 2, wherein the treatment scope of the treatment target is one third to two thirds of both sides of the perirenal fat tissue.

7. A method for treating essential hypertension by means of a high-intensity focused ultrasound system, comprising the following steps of:
(1) conducting ultrasonic positioning measurement on a treatment target, namely both sides of perirenal fat tissue of an essential hypertension patient, and measuring the sagittal length, the sagittal width, the horizontal width and the height of the perirenal fat tissue;
(2) setting a therapeutic window and corresponding parameters of the high-intensity focused ultrasound system according to weight, height and subcutaneous fat thickness parameters of the patient, so as to make local temperature of the perirenal fat tissue during treatment reach 40-70° C., treatment portions are both sides of the perirenal fat tissue, and a treatment scope is one third to all of the whole tissue;
(3) after setting the therapeutic window and the corresponding parameters, starting the high-intensity focused ultrasound system to treat one side of the perirenal fat tissue according to the set parameters; and
(4) after treating one side of the perirenal fat tissue, treating the other side of the perirenal fat tissue according to the same method and parameters.

8. The method according to claim 7, wherein the treatment target is both sides of lower perirenal fat tissue.

9. The method according to claim 7, wherein time for treating one side of the treatment target is 9-70 min.

10. The method according to claim 7, wherein local temperature of the treatment target is made to reach 45-60° C. during treatment.

11. The method according to claim 7, wherein the treatment scope of the treatment target is one third to two thirds of both sides of the perirenal fat tissue.

12. The application according to claim 4, wherein time for treating one side of the treatment target is 20-40 min.

13. The method according to claim 9, wherein time for treating one side of the treatment target is 20-40 min.
